(19) United States
(12) Patent Application Publication
(10) Pub. No.: US 2026/0241122 A1
(43) Pub. Date: Aug. 20, 2026

(54) ALIGNMENT INDICATOR FOR A NASAL SPRAY DISPENSER

(71) Applicants: Aarhus Universitet, Aarhus C (DK); Region Midtjylland, Viborg (DK)

(72) Inventors: Jesper HØJAGER FABECH BILLE, Skanderborg (DK); Milos Rasmus FUGLSANG NISSEN, Risskov (DK)

(73) Assignees: Aarhus Universitet, Aarhus C (DK); Region Midtjylland, Viborg (DK)

(21) Appl. No.: 19/161,068

(22) PCT Filed: Mar. 4, 2024
(86) PCT No.: PCT/EP2024/055621

§ 371 Date: Aug. 29, 2025

(30) Foreign Application Priority Data
Mar. 3, 2023 (EP) 23159915.0

(51) Int. Cl. A61M 15/08

(52) U.S. Cl. CPC A61M 15/085; A61M 2205/581; A61M 2205/582; A61M 2205/583; A61M 2205/586

(57) ABSTRACT

The present disclosure relates to a visual alignment indicator for a nasal spray dispenser or an adapter for a nasal spray dispenser, and a nasal spray guide adapter for holding a nasal spray dispenser, including the visual alignment indicator, while dispensing a fluid product into a nostril of a user. A spray guide adapter for holding a nasal spray dispenser includes a lower end and an elongated upper end for dispensing the nasal spray fluid into a nostril of a user, the spray guide adapter includes a housing having a dispenser opening for inserting and holding the lower end of the nasal spray dispenser, and an alignment indicator configured for providing a user of the nasal spray dispenser with feedback on the orientation of the elongated upper end of the nasal spray dispenser in the nostril of the user.

### CROSS-REFERENCE TO RELATED APPLICATIONS

The present application is the U.S. national phase entry of International Patent Application No. PCT/EP2024/055621, filed Mar. 4, 2024, which claims priority to European Patent Application No. 23159915.0, filed Mar. 3, 2023, the entire contents of both are hereby incorporated by reference.

### TECHNICAL FIELD

The present disclosure relates to an alignment indicator for a nasal spray dispenser. The present disclosure further relates to a nasal spray dispenser, and a nasal spray guide adapter for holding a nasal spray dispenser, comprising the visual alignment indicator, for dispensing a fluid product into a nostril of a user.

### BACKGROUND

The nasal route has emerged as a viable route for drug delivery, including vaccines. It offers a non-invasive, effective way to avoid gastrointestinal degradation and lung toxicity. It also seems to be a favorable way to bypass the obstacles to the blood-brain barrier allowing direct drug delivery to the central nervous system. On the other hand, the basal drug delivery route also offers several practical advantages from the patients' point of view, such as non-invasiveness, substantially painless, ease of drug delivery, and favorable tolerability profile. Further, it is convenient for patients in long-term therapies, including children.

Several approaches have been developed to deliver drugs in the nasal cavities, including drug delivery devices such as syringes, breathe-powered bi-directional nasal devices, droppers, pressurized meter dose inhalers, and sprayers. Out of all of these devices, nasal spray devices have become the desired choice due to their convenience, making them rank amongst the most commonly used drug delivery devices. These nasal spray devices are relatively simple systems that can be driven by propellants or hand-actuated pump systems, for example when the patient needs to use the device, the patient inserts the dispensing head of the spray device into the nostril and actuates the device to dispense a dose of a fluid product.

Despite its advantages, the efficacy of the drug delivery by the nasal spray devices can be highly patient-specific due to the lack of well-defined protocols and self-administration techniques. It has been shown that one of the major setbacks of these devices is related to the orientation of the spray device at the moment of the dose, and this orientation determines whether targeting the target zone is successful. It has been demonstrated that the administration angle (orientation) is a key factor in efficient drug delivery. Foo demonstrated that angles less than 30° can achieve around 90% deposition efficiency compared with angles around 40°-50° where efficiency was around 30-50%, even at 30°the deposition efficiency decreases from 90 to 75% indicating the importance or the correct angle (orientation) of the nasal spray device. Hence, the administration angle should be clarified and emphasized in the user instructions.

Several prior spray devices have attempted to address these limitations, for example, document US 2022/257885 describes a device for nasal delivery of fluid products which makes it possible to control the orientation of the device in the nostril. It comprises electronic components such as a camera for determining the spatial positioning of the nasal device inside a nostril. Although this device improves the nasal spray dispensing orientation, it is a complicated and expensive device. It may not be suitable for long-term use, or suitable for users such as children or elderly user where technology could be an obstruction than an advantage.

US 2019/160233 discloses a nasal administration device comprising an upper alignment tab to stabilize and position the nasal administration device against the bridge portion of a patient's nose. However, the user cannot obtain visual confirmation that the nasal administration device is oriented correctly.

CN208660119 U, US 2020/078543, WO 2014/165694, FR 3024655, and WO 02/85282 describe other prior art devices.

### SUMMARY

A purpose of the present disclosure is to provide a simpler, affordable and reliable solution to control the orientation of a nasal spray device (nasal spray dispenser) while in the nostril of a user such that application of the content of the nasal spray dispenser can be provided towards a target zone inside the nostril.

The present disclosure therefore relates to a visual alignment indicator for a nasal spray dispenser, the nasal spray dispenser comprising a lower end and an elongated upper end for dispensing nasal spray fluid into a nostril of a user. In embodiments, the visual alignment indicator comprising at least two elements, in embodiments, different visual elements, configured for defining at least one line of sight for the user, thereby providing the user of the nasal spray dispenser with feedback, in embodiments, visual feedback, on the orientation of the elongated upper end of the nasal spray dispenser in the nostril of the user. In embodiments, a feedback is provided to the user when two of the visual elements are visually aligned to the user along one line of sight.

The present disclosure further relates to nasal spray dispenser and a spray guide adapter for holding a nasal spray dispenser, e.g. a multi-dose nasal device comprising a reservoir containing several doses. The nasal spray dispenser is for example of the type comprising a lower end (reservoir) and an elongated upper end for dispensing the nasal spray fluid into a nostril of a user.

In embodiments, the spray guide adapter comprises a housing. The housing may comprise a dispenser opening for inserting and holding the lower end of the nasal spray dispenser. In embodiments, also an alignment indicator that advantageously is configured for providing a user of the nasal spray dispenser with feedback, in embodiments, a visual feedback, on the orientation of the elongated upper end of the nasal spray dispenser in the nostril of the user. Visual feedback regarding the orientation of the elongated upper end of the nasal spray dispenser in the nostril of the user can for example be provided by a visual alignment indicator comprising at least two different visual elements, in embodiments, configured for defining at least one line of sight for the user. A visual feedback can then be provided to the user when two of the visual elements are visually aligned to the user along one line of sight.

A major advantage is that the alignment indicator demonstrated herein in itself, for example by the structural features of the alignment indicator, can define one or more lines of sight to the user, such that the user of the nasal spray dispenser can verify with the user's own eyes that the orientation in the nostril is correct.

Examples of the presently disclosed alignment indicator, nasal spray dispenser and spray guide adapter can be seen in the appended figures. During use of the adapter the nasal spray dispenser is placed and held in the dispenser opening. The nasal spray dispenser including the alignment indicator is grabbed by the user and the elongated upper end of the nasal spray dispenser is placed in a nostril of the user, like the user would normally do if he/she used the nasal spray dispenser alone. Once inside the nostril the nasal spray dispenser can be moved as in a cone, but the alignment indicator provides a feedback to user regarding the correct orientation.

In embodiments, the alignment indicator is a visual alignment indicator such that the feedback to the user regarding the orientation inside the nostril is visual.

As exemplified in the figures the alignment indicator can be formed as a channel, for example at least partly or fully through the housing of the spray guide adapter, and the orientation of the channel relative to the orientation of the nasal spray dispenser defines the application point of the nasal spray inside the nostril of the user, because the user will orient the nasal spray adapter such that he/she looks through or into the channel of the alignment indicator. The diameter of the channel can be made thin, e.g. few mm or even less than 1 mm, such that only when the user can see through, or see light through or in the channel, the visual feedback about orientation is provided. Alternatively a cross-hair pattern inside the channel can provide additional accuracy as part of the alignment indicator as for example illustrated in FIG. 10, this would typically require a channel of more than 3-5 mm in diameter.

The presently disclosed alignment indicator and related dispenser and spray guide adapter therefore provide for an easy, affordable and reliable method to ensure precise angular positioning of the nasal spray dispenser at the moment a nasal spray dose is dispensed, thereby assuring optimal deposition of the fluid product which is dispensed into the nostril.

In particular because the alignment indicator provides the necessary feedback to the user while dispensing the dose.

The presently disclosed visual alignment indicator, dispenser and spray guide adapter provide a solution that can be used with a large number of nasal spray dispensing devices of the same type, and is thus not limited to the examples described herein. The presently disclosed alignment indicator and spray guide adapter does not have any impact on the operation and performance of the nasal spray dispenser, and thus does not modify the efficacy of the fluid product which is dispensed.

The presently disclosed solution can be used in any position of the user, in particular standing, sitting, lying down, and embodiments of the alignment indicator, dispenser and adapter do not require any electronics, such as cameras or lasers to initially obtain the optimal orientation in the nostril to dispense a dose in the optimal position. The presently disclosed alignment indicator, dispenser and spray guide adapter therefore provide a convenient solution for patients in long-term therapies including children.

Evidently, the alignment indicator, the dispenser and the nasal spray adapter are not limited to the multi-dose device described above, but applies to any nasal spray dispenser device, whether it be a single-dose device, a two-dose device, or a multi-dose device. The presently disclosed solution is particularly intended for the treatment of any pathology which can be treated nasally requiring precise orientation of the nasal dispenser such as, for example, vaccination.

As stated above, the presently disclosed alignment indicator can be part of the nasal spray dispenser such that the alignment indicator is fixed to and/or is an integrated part of the nasal spray dispenser. In that regard the present disclosure further relates to nasal spray dispenser (as described herein) comprising the alignment indicator as disclosed herein, in embodiments, as a fixed and/or integral part of the nasal spray dispenser. For example such that the alignment indicator is configured for providing a user of the nasal spray dispenser with feedback on the orientation of the elongated upper end of the nasal spray dispenser in the nostril of the user. In embodiments, the alignment indicator is a visual alignment indicator configured for defining a line of sight, e.g. for at least one eye of the user, for providing a visual feedback to the user.

### DESCRIPTION OF DRAWINGS

FIG. 1a shows an upper view of one embodiment of the presently disclosed spray guide adapter.

FIG. 1b shows a cross-sectional view of one embodiment of the presently disclosed spray guide adapter.

FIG. 1c shows a side view of one embodiment of the presently disclosed spray guide adapter.

FIG. 1d shows a perspective view of one embodiment of the presently disclosed spray guide adapter.

FIG. 2a shows a lower view of one embodiment of the presently disclosed spray guide adapter.

FIG. 2b shows a front perspective view of one embodiment of the presently disclosed spray guide adapter showing.

FIG. 2c shows a rear perspective view of one embodiment of the presently disclosed spray guide adapter.

FIG. 3a shows a rear view of one embodiment of the presently disclosed spray guide adapter.

FIG. 3b shows a front view of one embodiment of the presently disclosed spray guide adapter.

FIG. 3c shows a rear perspective view of the presently disclosed spray guide adapter.

FIG. 3d shows a front perspective view of one embodiment of the presently disclosed spray guide adapter.

FIG. 4a shows an upper view of one embodiment of the presently disclosed spray guide adapter in a foldable format before folding.

FIG. 4b shows a lower view of one embodiment of the presently disclosed spray guide adapter in a foldable format before folding.

FIG. 4c shows a front view of one embodiment of the presently disclosed spray guide adapter in a foldable format before folding.

FIG. 4d shows a side view of one embodiment of the presently disclosed spray guide adapter in a foldable format before folding.

FIG. 4e shows a rear view of one embodiment of the presently disclosed spray guide adapter in a foldable format before folding.

FIG. 4f shows an upper perspective view of one embodiment of the presently disclosed spray guide adapter in a foldable format before folding.

FIG. 4g shows a lower perspective view of one embodiment of the presently disclosed spray guide adapter in a foldable format before folding.

FIG. 5a shows an upper view of one embodiment of the presently disclosed spray guide adapter in a foldable format after folding.

FIG. 5b shows a lower view of one embodiment of the presently disclosed spray guide adapter in a foldable format after folding.

FIG. 5c shows a front view of one embodiment of the presently disclosed spray guide adapter in a foldable format after folding.

FIG. 5d shows a side view of one embodiment of the presently disclosed spray guide adapter in a foldable format after folding.

FIG. 5e shows a rear view of one embodiment of the presently disclosed spray guide adapter in a foldable format after folding.

FIG. 5f shows an upper perspective view of one embodiment of the presently disclosed spray guide adapter in a foldable format after folding.

FIG. 5g shows a lower perspective view of one embodiment of the presently disclosed spray guide adapter in a foldable format after folding.

FIG. 6a shows a front perspective view of one embodiment of the presently disclosed spray guide adapter having an adjustable dispensing angle.

FIG. 6b shows an upper view of one embodiment of the presently disclosed spray guide adapter having an adjustable dispensing angle.

FIG. 6c shows a rear perspective view of one embodiment of the presently disclosed spray guide adapter having an adjustable dispensing angle.

FIG. 6d shows a front view of one embodiment of the presently disclosed spray guide adapter having an adjustable dispensing angle.

FIG. 6e shows a side view of one embodiment of the presently disclosed spray guide adapter having an adjustable dispensing angle.

FIG. 6f shows a rear view of one embodiment of the presently disclosed spray guide adapter having an adjustable dispensing angle.

FIG. 6g shows a front perspective view of one embodiment of the presently disclosed spray guide adapter having an adjustable dispensing angle.

FIG. 6h shows a lower view of one embodiment of the presently disclosed spray guide adapter having an adjustable dispensing angle.

FIG. 6i shows a rear perspective view of one embodiment of the presently disclosed spray guide adapter having an adjustable dispensing angle.

FIG. 7a shows a front top perspective view of one embodiment of the presently disclosed spray guide adapter holding a nasal spray dispenser.

FIG. 7b shows a cross-sectional view of one embodiment of the presently disclosed spray guide adapter holding a nasal spray dispenser.

FIG. 7c shows a rear top perspective view of one embodiment of the presently disclosed spray guide adapter holding a nasal spray dispenser.

FIG. 7d shows a front view of one embodiment of the presently disclosed spray guide adapter holding a nasal spray dispenser.

FIG. 7e shows a side view of one embodiment of the presently disclosed spray guide adapter holding a nasal spray dispenser.

FIG. 7f shows a rear view of one embodiment of the presently disclosed spray guide adapter holding a nasal spray dispenser.

FIG. 7g shows a front lower perspective view of one embodiment of the presently disclosed spray guide adapter holding a nasal spray dispenser.

FIG. 7h shows a rear lower perspective view of one embodiment of the presently disclosed spray guide adapter holding a nasal spray dispenser.

FIG. 8a shows the use of one embodiment of the presently disclosed spray guide by showing the insertion of the nasal spray dispenser into the nostril.

FIG. 8b shows the use of one embodiment of the presently disclosed spray guide by showing the lateral orientation of the nasal device in the nostril.

FIG. 9a shows a front perspective view of one embodiment of the presently disclosed spray guide adapter, wherein the alignment indicator comprises different visual symbols “R” and “L”, such that the adapter is suitable for correct orientation in the both left and right nostrils of the user.

FIG. 9b shows another front perspective view of one embodiment of the presently disclosed spray guide adapter, wherein the alignment indicator comprises different visual symbols “R” and “L”, such that the adapter is suitable for correct orientation in the both left and right nostrils of the user.

FIG. 9c shows another front perspective view of one embodiment of the presently disclosed spray guide adapter, wherein the alignment indicator comprises different visual symbols “R” and “L”, such that the adapter is suitable for correct orientation in the both left and right nostrils of the user.

FIG. 9d shows another front perspective view of one embodiment of the presently disclosed spray guide adapter, wherein the alignment indicator comprises different visual symbols “R” and “L”, such that the adapter is suitable for correct orientation in the both left and right nostrils of the user.

FIG. 9e shows another front perspective view of one embodiment of the presently disclosed spray guide adapter, wherein the alignment indicator comprises different visual symbols “R” and “L”, such that the adapter is suitable for correct orientation in the both left and right nostrils of the user.

FIG. 9f shows another front perspective view of one embodiment of the presently disclosed spray guide adapter, wherein the alignment indicator comprises different visual symbols “R” and “L”, such that the adapter is suitable for correct orientation in the both left and right nostrils of the user.

FIG. 10a shows a side view of one embodiment of the presently disclosed nasal spray dispenser with the presently disclosed alignment indicator as an integral part of the dispenser, wherein the alignment indicator comprises a front sight.

FIG. 10b shows a top perspective view of one embodiment of the presently disclosed nasal spray dispenser with the presently disclosed alignment indicator as an integral part of the dispenser, wherein the alignment indicator comprises a front sight.

FIG. 10c shows a front perspective view of one embodiment of the presently disclosed nasal spray dispenser with the presently disclosed alignment indicator as an integral part of the dispenser, wherein the alignment indicator comprises a front sight.

FIG. 10d shows a partial sectional view of one embodiment of the presently disclosed nasal spray dispenser with the presently disclosed alignment indicator as an integral part of the dispenser, wherein the alignment indicator comprises a front sight.

FIG. 10e shows a partial perspective view of one embodiment of the presently disclosed nasal spray dispenser with the presently disclosed alignment indicator as an integral part of the dispenser, wherein the alignment indicator comprises a front sight.

FIG. 10f shows a lower perspective view of one embodiment of the presently disclosed nasal spray dispenser with the presently disclosed alignment indicator as an integral part of the dispenser, wherein the alignment indicator comprises a front sight.

### DETAILED DESCRIPTION

The presently disclosed solution provides a for reliable method to ensure that the required angular positioning of the nasal spray dispenser is achieved the moment a dose is dispensed. The presently disclosed approach relies on an alignment indicator to provide feedback to the user while dispensing the dose. In embodiments, the footprint of the visual alignment indicator and the spray guide adapter can be quite small, with height (or the housing thereof) of possibly less than 40 mm, less than 30, or less than 20 mm. In embodiments, the width of the spray guide adapter is between less than 40, 30, or 20 mm. In embodiments, the length of the spray guide adapter is less than 40, 30, or 20 mm.

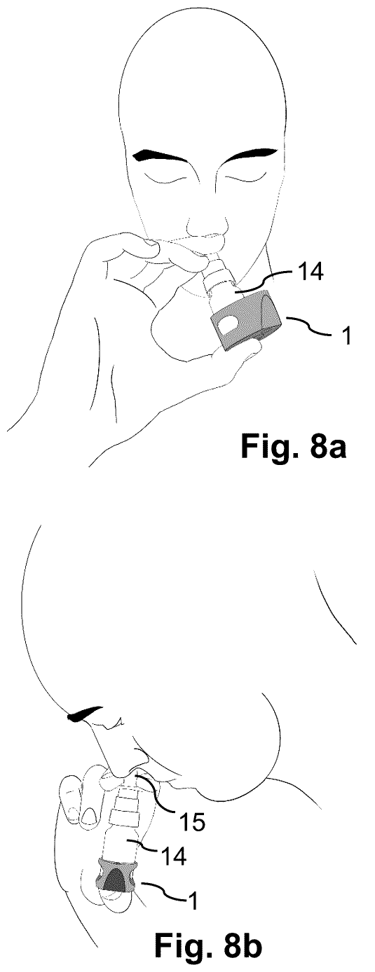

In embodiments, the alignment indicator is a visual alignment indicator configured for providing visual feedback to the user. In embodiments, the visual alignment indicator defines a line of sight, i.e. a line of sight to the user during use of the nasal spray dispenser held by the adapter. In particular, the alignment indicator may be configured to be a front sight for the user while orienting the upper end of the nasal spray dispenser in the nostril while holding the nasal spray adapter or dispenser. See for example FIGS. 8a and 8b where a user places the upper end of the spray dispenser into the nostril while looking through a visual alignment indicator.

An embodiment relates to a spray guide adapter for holding a nasal spray dispenser comprising a lower end and an elongated upper end for dispensing nasal spray fluid into a nostril of a user, the spray guide adapter, optionally comprising a housing, comprising a dispenser opening for inserting and holding the lower end of the nasal spray dispenser, and a visual alignment indicator comprising at least two different visual elements configured for defining at least one line of sight for the user, thereby providing the user of the nasal spray dispenser with visual feedback on the orientation of the elongated upper end of the nasal spray dispenser in the nostril of the user, wherein a visual feedback is provided to the user when two of the visual elements are visually aligned to the user along one line of sight.

In one embodiment the alignment indicator forms an axis, e.g. defined by the extension of the channel, that is non-parallel with an axial direction formed by the dispenser opening, such that when the nasal spray dispenser is held by the dispenser opening, a longitudinal axis formed by the elongated upper end of the nasal spray dispenser forms an alignment angle with a line of sight of the alignment indicator. Hence, the alignment indicator may form an axis which is non-parallel with a longitudinal axis formed by the elongated upper end of the dispenser, such that said longitudinal axis forms an alignment angle with a line of sight of the alignment indicator.

This alignment angle is provided to ensure the correct application angle of the spray dispenser in the nostril of the user. In embodiments, the alignment angle is more than 0 degrees, less than 40 degrees, less than 30 degrees, less than 25 degrees, or less than 20 degrees. In embodiments, the angle is between 2 and 40 degrees, between 5 and 30 degrees, or between 5 and 20 degrees, for example, 15 degrees as in the example in some of the figures.

There are different ways of defining a line of sight to the user as part of the visual alignment indicator. One approach is to separate the different visual elements along an axis parallel with the line of sight. In one embodiment the visual alignment indicator therefore extends longitudinally between at least two different visual elements. For example at least one of the visual elements may be located towards an upper part of the adapter and at least one other and different visual element is located towards a lower part of the adapter. This is illustrated in the different embodiments in the drawings.

Hence, in one embodiment at least two of the different visual elements, such as a hollow element and a solid element, are separated along the line of sight and/or separated along a longitudinal axis defined by the nasal spray dispenser. In embodiments, the two different visual elements may be separated by at least 10 mm, by at least 20 mm, by at least 30, or by at least 40 mm. In the illustrated embodiments in the drawings this separation distance is more or less defined by the height of the adapter.

In one embodiment the visual elements of the alignment indicator comprise at least one solid element and at least one hollow element. A similar effect provided with a hollow and a solid visual element can be obtained if one of the visual elements are transparent, for example such that one visual element can be seen through the other visual element. E.g. an upper visual element may be transparent and the like of sight and the visual feedback to user is provided when a lower visual element is visible through the upper transparent visual element.

Hence, in one embodiment a visual feedback is provided when one of the visual elements, such as a solid visual element, is visible to the user through another of the visual elements, such as a hollow and/or transparent visual element.

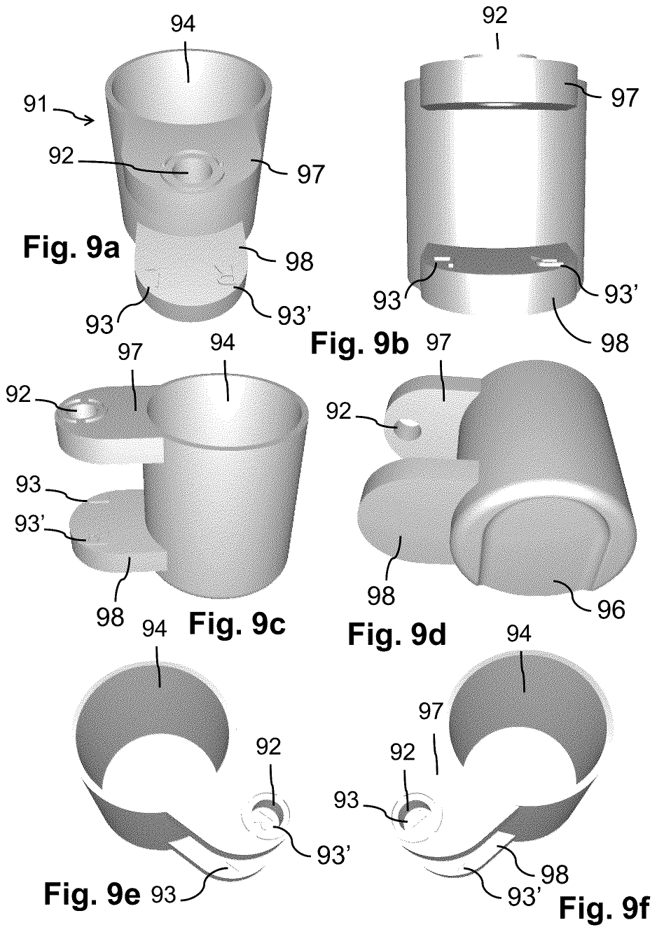

In particular the visual elements may comprise at least two solid elements and at least one hollow element. The two solid elements may be different, such as form different but recognizable symbols, such as different letters, like “R” and “L”, or different numbers. This for example illustrated in FIGS. 9a-9b, showing an embodiment with an upper hollow visual element in the form of a channel/opening and two lower visual elements formed as the letters “R” and “L”.

The visual elements may comprise at least two solid elements. At least two of the visual elements, such as two solid elements and/or two hollow elements, may be separated in a plane defined by a bottom end of the adapter or a plane defined by a bottom end of the dispenser. See for example the embodiment in FIGS. 9a-9f. In embodiments, the visual elements may be separated in the plane by at least 5 mm, by at least 10 mm, by at least 15, or by at least 20 mm.

Such a solution can be used when the visual alignment indicator is configured for defining two different lines of sight for the user. This is in particular useful when the two different lines of sight are configured for orientation in the left nostril of the user and in the right nostril of the user, respectively. Hence, the visual alignment indicator may be configured for visually indicating a first correct orientation in the left nostril of the user and a second correct orientation in the right nostril of the user.

In one embodiment the visual alignment indicator comprises at least two elements, for example an upper element and a lower element, extending radially from the dispenser/dispenser opening/housing. The visual elements may correspondingly be formed in said elements. See for example the embodiment in FIGS. 9a-9f, where two radially extending elements are provided to house a see-through opening in the upper element and two different solid elements in the form letters in the lower element.

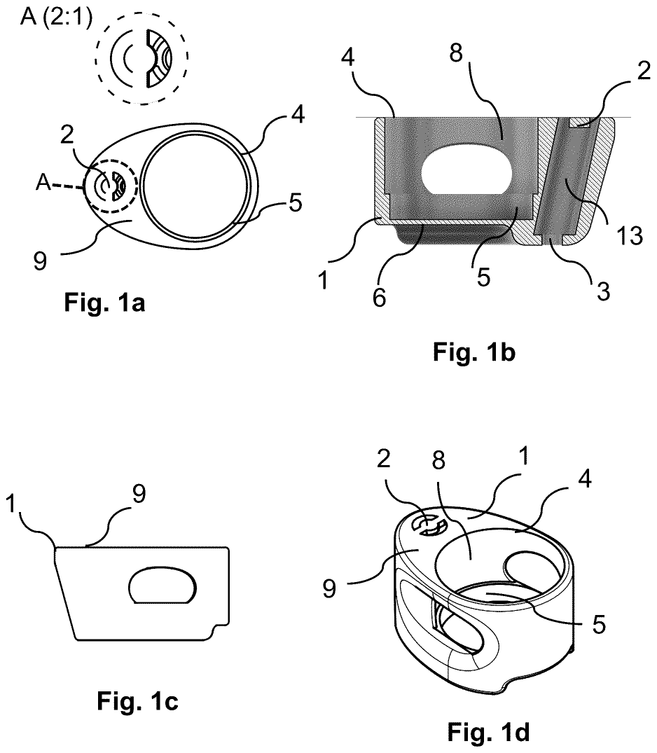

As stated above, the alignment indicator may comprise at least two different visual elements, such as a solid element and a hollow element, which can be located inside a channel extending through the housing, e.g. from the upper end to the lower end of the housing. The solid element of the alignment indicator can be a solid circle, and/or wherein the second (hollow) element can be a circular ring. The solid circle is located towards the top surface of the adapter, and the circular ring is located towards the bottom surface of the adapter (or vice versa) and these two elements are separated by a distance. In that way the user orients the housing of the spray guide adapter such that the two visual elements are aligned to the eyes of the user while looking through the alignment indicator, for example, the solid circle is within the circular ring. The two elements of the visual alignment are in a channel that is non-parallel to and displaced from the dispenser opening as shown in FIGS. 1 a-1d. In embodiments, the diameter of the channel is less than 10 mm, such as less than 8 mm or less than 5 mm. In embodiments, the diameter of the channel is less than 3, 2 or 1 mm, for example if the channel itself provides the visual alignment.

The alignment indicator may be provided as other solutions, for example, a light source that will turn on as the adapter hits the correct position or a sensor configured for providing a tactile signal, such as a sound and/or vibration as the adapter is on the correct angle. Possibly even a light source at the bottom of the channel. The alignment indicator could also be configured to provide a visual signal, such as a word or image, which will appear when the adapter is within a predefined spatial orientation.

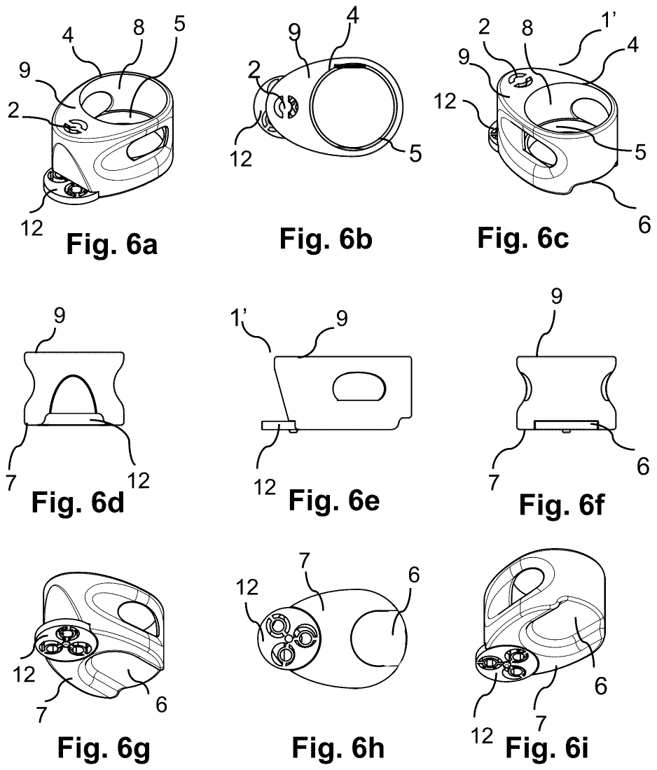

The alignment indicator may be adjustable, for example, the bottom element of the alignment indicator may have at least two or at least three different options, which for example can be chosen by rotating a disk, as shown in an example in FIGS. 6a-6i. These different alignment options offer the possibility to target different areas of the nostril, for example, one option may be aiming for the tuba osmium, the other may aim at the middle meatus and the third option could aim toward the meatus superior (frontal osmium). The option to reach different respiratory regions allows users to administer different drugs with different requirements, therefore treat different diseases. For example, the drug delivered to the meatus superior area has a longer retention time, which is beneficial for the absorption of drugs for systemic effect. The meatus superior or olfactory region has been shown to have the potential to transport the drug to the brain and is the targeted absorption area of drugs for the therapy of central nervous diseases. The middle meatus is the best-targeted position for the therapy of sinusitis disorders. For nasal vaccination, it is important to target the target deposition area with the higher immunogenic responses.

In one embodiment of the adapter the dispenser opening is located at the upper end of (the housing of) the nasal spray adapter. This opening forms a casing for the lower end of the nasal spray dispenser, it starts from the upper end of the adapter to near the bottom end of the adapter as shown in FIGS. 1a-1d. The dispenser opening may comprise a protrusion that is located within an inner layer near the bottom end of the opening. The protrusion is extended towards the center of the opening and it functions as a securing feature to hold the lower end of the nasal spray dispenser, like a snap-fit, while the user is dispensing the dose of the nasal fluid.

In one embodiment the nasal spray adapter (and/or the dispenser) comprises an indentation for a finger, such as a thumb, of the user. The indentation is used as a support guiding mechanism to help the user to grip the dispenser/adapter in the correct position. In embodiments, the indentation is located in the bottom end of (the housing of) the nasal spray dispenser/adapter and it will help the user to hold the dispenser/adaptor in the correct position regardless if the users grab the nasal spray guide adapter/dispenser with the left or right hand, see for example FIGS. 8a-8b. As stated herein, such an indentation can also be part of the dispenser itself, if for example the alignment indicator is an integral part of the dispenser.

The dispenser, the alignment indicator and/or the nasal spray adapter may be manufactured using different materials and processes for example it can be made of polymer/plastic, metal, or cardboard. In embodiments, the dispenser, the adapter and/or the alignment indicator can be manufactured using injection molding to lower the cost of mass production. The dispenser, the nasal spray guide adapter and/or the alignment indicator can be also 3D printed. If provided as a cardboard solution, possibly foldable, the adapter can be incorporated in the spray dispenser's packaging which is commonly made of cardboard. If incorporated in the cardboard packaging the adapter will typically be flat, but after separation from the packaging the adapter can be folded and used by the user. In a foldable solution the alignment indicator can be formed by two holes in the housing, and when folded the two holes will be rotated. Across each other and the location of the two holes will define the alignment angle and the line of sight of the alignment indicator. The holes can be provided with pattern(s), such as a cross-hair pattern, for increased accuracy in the alignment.

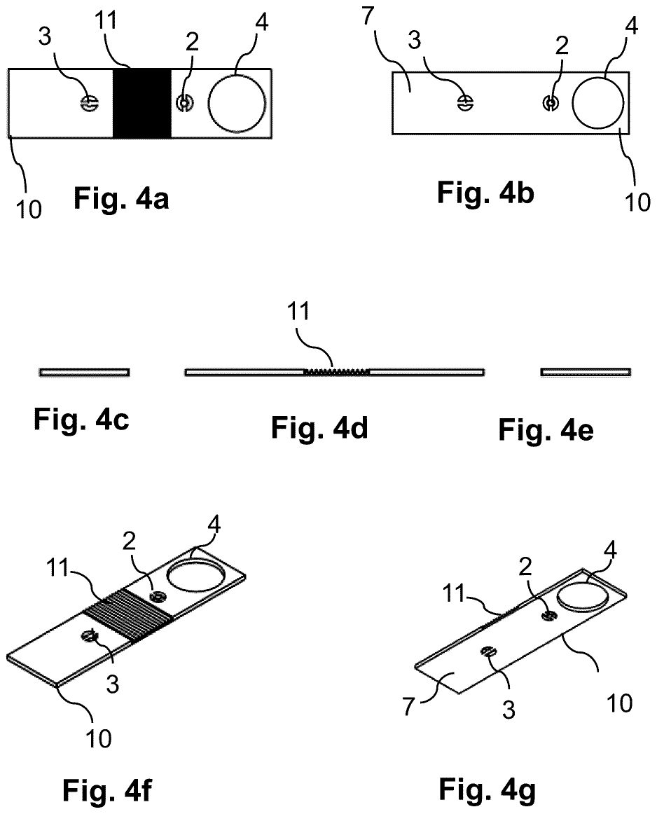

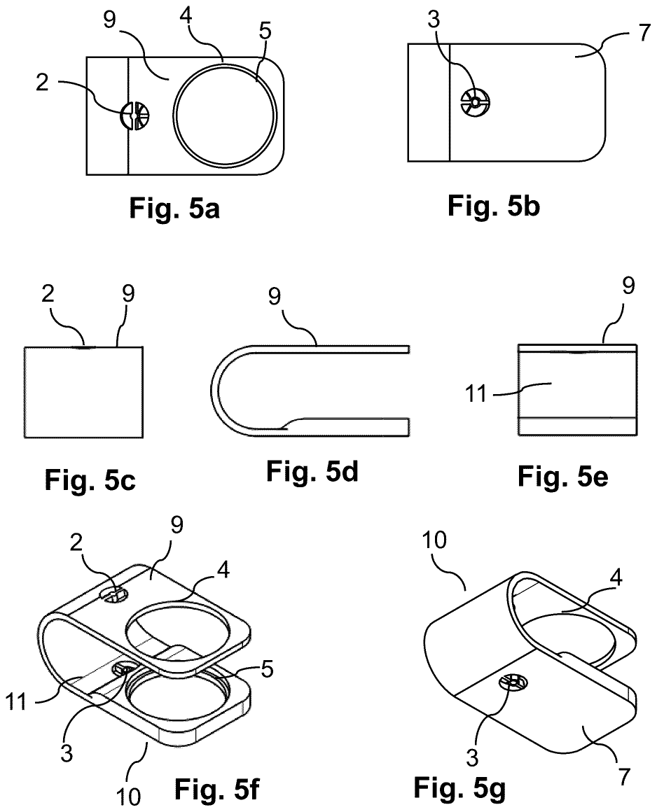

Hence, the nasal spray guide adapter may be in a foldable form as shown in FIGS. 5a-5g. Here, the nasal spray guide adapter is provided as a substantially plane element with visible folding lines indicating the folding direction as shown in FIGS. 4a-4g. Here the unfolded nasal spray guide adapter can easily be packaged alongside the nasal spray dispenser, or even as part of the packaging as mentioned above.

In one embodiment the fluid of the nasal spray dispenser is selected from the group of antihistamines, corticosteroids, saline, topical decongestants, and nasal vaccine.

The present disclosure further relates to a method of guided nasal spraying in a nostril of a user from a nasal spray dispenser comprising the steps of:
Placing the lower end of a nasal spray dispenser in the dispenser opening of the presently disclosed spray guide adapter, or
providing a nasal spray dispenser having the presently disclosed visual alignment indicator attached/fixed thereto, in embodiments, as an integral part;
Grabbing by the user the adapter with the nasal spray dispenser attached or grabbing the nasal spray dispenser with the alignment indicator,
Inserting, by the user, the elongated upper end of the nasal spray dispenser inside the nostril of the user;
Angularly orientating by the user, while holding the nasal spray dispenser, the elongated upper end of the nasal spray dispenser inside the nostril of the user until the alignment indicator is aligned for the user by a visual and/or tactile feedback, and
Activating the nasal spray dispenser to deliver nasal spray to the nostril of the user.

In a particular embodiment it is possible to adjust the dispensing angle of fluid of the nasal spray dispenser by changing the position and/or the line of sight of the alignment indicator as shown in FIGS. 6a-6i. As the angle is adjusted and the user has inserted the elongated upper end of the nasal spray dispenser inside their nostril, then the alignment indicator is aligned visually by aligning two elements of a cross-hair pattern.

The present disclosed solution is not limited to holding multi-dose dispensers, because it can be applied to any fluid product or powder dispensing device either of the single-dose type, i.e. comprising a reservoir that contains a single dose which is dispensed in a single actuation, of the two-dose type, i.e. comprising a reservoir which contains two doses which are dispensed in two successive actuations, or of the multi-dose type, i.e. comprising a reservoir which contains more than two doses

### DETAILED DESCRIPTION OF DRAWINGS

In the description below, the terms “top surface”, “bottom end”, refer to the upright position of the dispensers and adapters shown in the drawings.

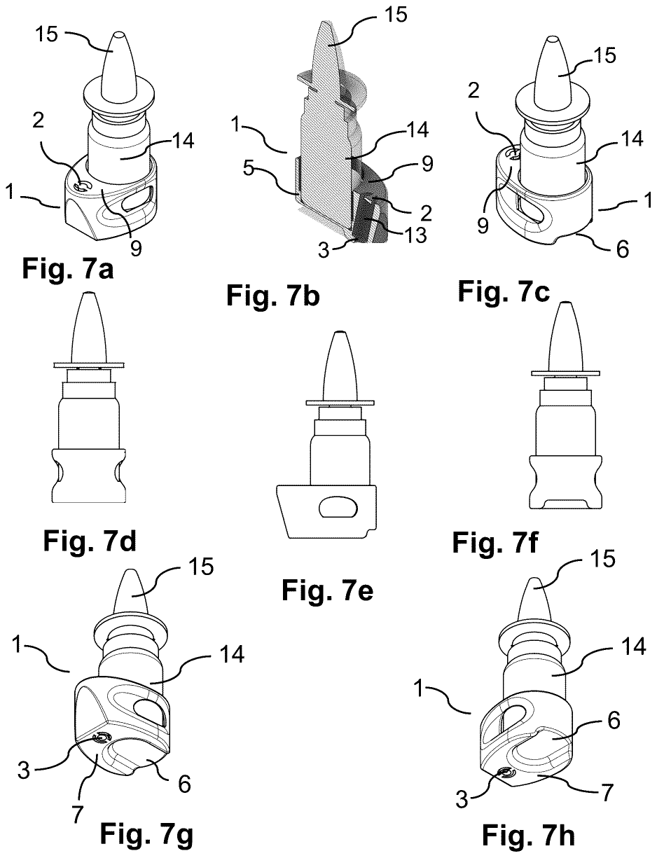

FIGS. 1a-1d shows one embodiment of the nasal spray guide adapter 1 using different viewpoints: an upper view (FIG. 1a), a cross-section view (FIG. 1b), a side view (FIG. 1c), and an overall 3D schematic (FIG. 1d). FIG. 1a shows the upper view with some features of the nasal spray guide adapter with the top surface 9, the solid circle element 2 which is the upper element of the alignment indicator, and a close-up A of the solid circle. The dispenser opening 4 and the protrusion 5 are provided to insert and hold the lower end 14 of the nasal spray dispenser 14, as seen in FIGS. 7a-7h. FIG. 1b is a cross-section of the nasal spray guide adapter 1 showing the opening 4, the inner layer 8 of the nasal spray guide adapter, the protrusion 5, and both elements 2, 3 that configure the visual alignment indicator, as well as the channel 13 connecting these two elements 2, 3. FIG. 1b also shows the indentation for thumb 6. FIG. 1c is a side view of the nasal spray guide adapter 1 showing its top surface 9. FIG. 1d is a 3D schematic of the nasal spray guide adapter showing its top surface 9, the solid circle 2 element of the alignment indicator, the dispenser opening 4, the inner layer 8 of the nasal spray guide adapter 8, and the protrusion 5.

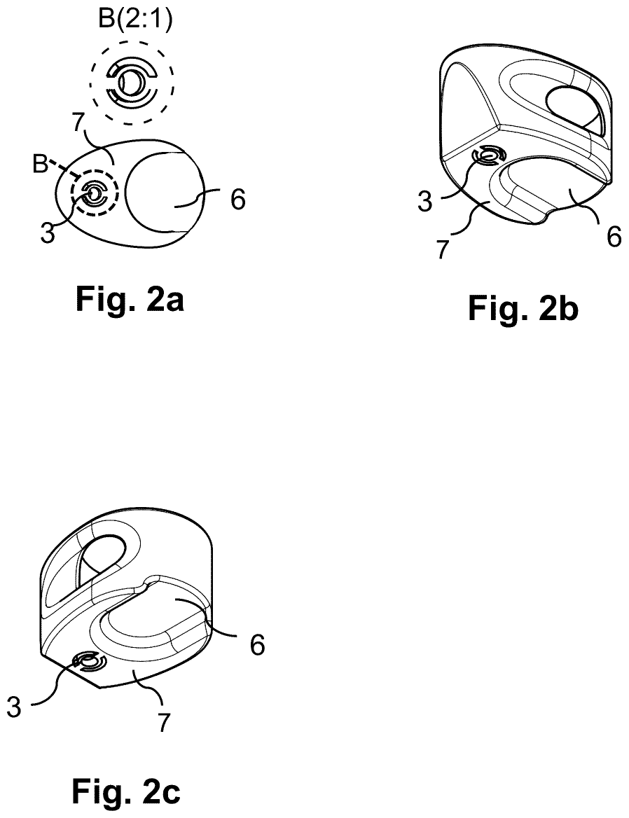

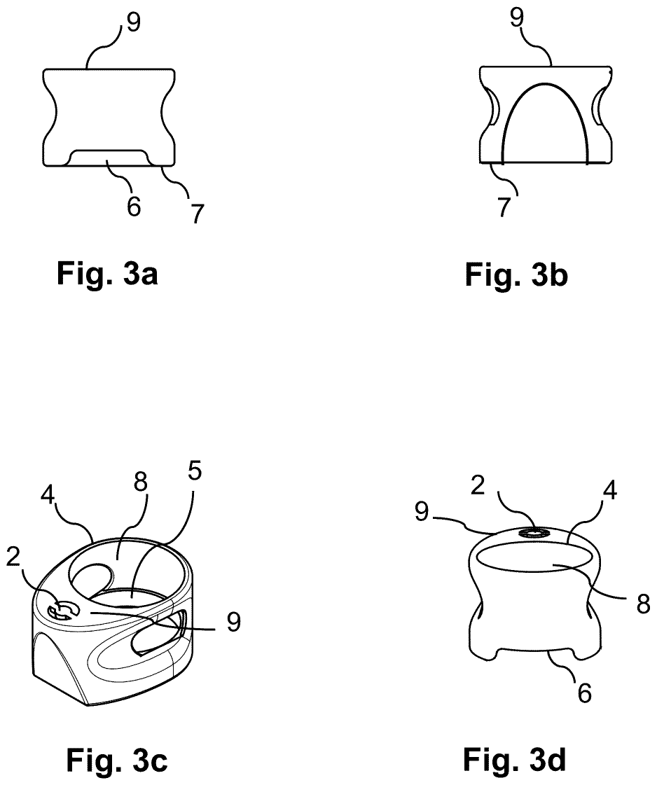

FIGS. 2a-2c shows the lower features of the nasal spray guide adapter 1 of FIG. 1. It shows illustrations from the lower view (FIGS. 2a) and 3D schematics from the side and back (FIG. 2b and FIG. 2c respectively) of the nasal spray guide adapter 1. Here in FIG. 2a the thumb indentation 6, i.e. the feature that can guide and help the user to grab the nasal spray guide adapter 1, and the bottom end 7 of the nasal spray guide adaptor are shown. FIG. 1a also shows the hollow lower element 3 of the visual alignment indicator as well as a close-up B of the hollow element 3.

FIGS. 3a-3d displays a back view (FIG. 3a), and front view (FIG. 3b), and 3D schematics from the back and front view (FIG. 3c and FIG. 3d, respectively) of the nasal spray guide adapter 1 seen in FIGS. 1-2. FIG. 3a shows the top surface 9, the indentation 6 for a thumb, and the bottom end 7 of the nasal spray guide adaptor 1. FIG. 3b shows the top surface 9 and the bottom end 7 of the nasal spray guide adaptor. FIG. 3c is a 3D schematic of the nasal spray guide adapter showing its top surface 9, the solid circle element 2 of the alignment indicator, the dispenser opening 4, the inner layer 8 of the nasal spray guide adapter 1, and the protrusion 5. FIG. 3d is a 3D schematic of the nasal spray guide adapter 1 showing its back showing the top surface 9, the solid circle element 2 of the alignment indicator, the dispenser opening 4, the inner layer 8 of the nasal spray guide adapter 1 and the indentation 6 for a thumb.

FIGS. 4a-4g and FIGS. 5a-5g shows an embodiment of a foldable spray guide adapter 10. FIGS. 4a-4g shows the foldable nasal spray guide adapter 10 in unassembled/unfolded form. FIGS. 4a-4g includes the upper view (FIG. 4a), lower view (FIG. 4b), front view (FIG. 4c), side view (FIG. 4d), and back view (FIG. 4e). FIGS. 4f and 4g also includes 3D schematics from the upper and lower view (respectively) of the foldable nasal spray guide adapter 10. FIG. 4a shows the upper view having folding lines 11, the two elements 2, 3 of the visual alignment indicator, and the dispenser opening 4. FIG. 4b shows the lower view of the unfolded nasal spray guide adapter 10 having the two elements 2, 3 of the visual alignment indicator and the dispenser opening 4. FIG. 4c shows the front view of the unfolded adapter 10. FIG. 4d shows the side view of the unfolded adapter 10 as well as the folding line 11. FIG. 4 e shows the front view of the unfolded adapter 10 having the two elements 2, 3 of the visual alignment indicator and the dispenser opening 4. FIG. 4f is a 3D schematic of the upper view of the unfolded nasal spray guide adapter 10 showing both the solid circle element 2 and the lower hollow element 3 of the alignment indicator, the dispenser opening 4, and the folding lines 11. FIG. 4g is a 3D schematic of the lower view of the unfolded nasal spray guide adapter 10 showing both the solid circle element 2 and the lower element 3 of the visual alignment indicator, the dispenser opening 4, and the folding lines 11.

FIGS. 5a-5g shows the foldable nasal spray guide adapter 10 in folded form where it is ready for use, i.e. for receiving and holding the dispenser. FIG. 5a shows the upper view with some features of the foldable nasal spray guide adapter such as the top surface 9, the solid circle element 2 which is the upper element of the alignment indicator, the dispenser opening 4, and the protrusion 5. FIG. 5b shows the lower view of the folded nasal spray guide adapter 10, having the lower ring element 3 of the visual alignment indicator and the bottom end 7 of the folded nasal spray guide adaptor 10. FIG. 5c shows the front view of the folded adapter 10 with features such as the top surface 9 and the solid circle element 2 which is the upper element of the alignment indicator. FIG. 5d shows the side view of the unfolded adapter 10 with the top surface 9. FIG. 5e shows the back view of the folded adapter 10 having the top surface 9 and the folding lines 11. FIG. 5f is a 3D schematic of the upper view of the folded nasal spray guide adapter 10 showing the top surface 9, both the solid circle element 2 and the lower hollow element (ring) of the visual alignment indicator, the dispenser opening 4, the protrusion 5 and the folding lines 11. FIG. 5g is a 3D schematic of the lower view of the folded nasal spray guide adapter 10 showing the lower hollow element 3 (ring) of the visual alignment indicator, the dispenser opening 4 and the bottom end 7 of the folded adapter 10.

FIG. 6a-6i shows one embodiment of the nasal spray guide adapter 1′ wherein the visual alignment indicator is adjustable, i.e. such that the target application angle of the nasal spray can be adjusted. FIG. 6a is a 3D schematic of the nasal spray guide adapter 1′ from the front view showing its top surface 9, the solid circle element 2 which is the upper element of the alignment indicator, the dispenser opening 4, the protrusion 5, the inner layer 8 of the nasal spray guide adapter 1′, and the rotational element 12 that allows the user to have three different angles. FIG. 6b shows the upper view of the nasal spray guide adaptor 1, with the top surface 9, the solid circle element 2 which is the upper element of the alignment indicator, the dispenser opening 4, the protrusion 5, and the rotational element 12. FIG. 6c is a 3D schematic of the nasal spray guide adapter 1′ from the back view also showing the indentation 6 for a thumb. FIG. 6d is a front view of the nasal spray guide adapter 1′. FIG. 6e is a side view of the nasal spray guide adapter 1′, and FIG. 6f is a back view of the nasal spray guide adapter 1′. FIG. 6g is a 3D schematic of the nasal spray guide adapter 1′ from the front view showing its bottom surface 7, the rotational element 12 of the visual alignment indicator, and the indentation 6 for a thumb. FIG. 6h is a lower view of the nasal spray guide adaptor 1′ and FIG. 6i is a 3D schematic of the nasal spray guide adapter 1′ from the back view. As seen in FIGS. 6a-6i the rotational element 12 comprises three versions of the lower hollow element forming part of the alignment indicator. The rotational element 12 can be rotated such that each of the three hollow element can be brought into the longitudinal channel of the alignment indicator. The three hollow elements of the rotational element 12 have different distances to the rotational axis of the rotational element 12 such that target application angle of the corresponding nasal spray dispenser will be different depending on which of the three hollow elements of the rotational element that is selected.

FIGS. 7a-7h shows the nasal spray guide adapter 1 of FIGS. 1a-3d with the lower end 14 of the nasal spray dispenser held by the adapter 1 in the dispenser opening 4 with the upper end 15 of the dispenser pointing up. FIG. 7a shows a 3D schematic of the nasal spray guide adapter 1 and the dispenser, FIG. 7b is a cross-sectional view of the nasal spray guide adapter 1 holding the nasal spray dispenser where the channel 13 of the alignment indicator is visible, and where it is clearly visible that this channel 13 is non-parallel to the extension of the upper end 15 of the dispenser, and the angle formed between the dispenser and the channel defines the target application angle, in this example the angle is 15 degrees. FIG. 7 c shows a 3D schematic of the nasal spray guide adapter 1 from the back view, FIG. 7d shows a front view of the nasal spray guide adapter 1 holding the nasal spray dispenser, FIG. 7e is a side view, and FIG. 7f is a back view. FIG. 7g is a 3D schematic front view and FIG. 7h is a 3D schematic back view.

FIGS. 8a-8b shows use of the nasal spray guide adapter 1, showing the insertion of the upper end 14 of the nasal spray dispenser into the nostril (FIG. 8a) and the lateral orientation in the nostril (FIG. 8b). As seen from FIGS. 8a-8b the correct positioning of the spray guide adapter 1 is achieved by the handling of the adapter 1 by placing the thumb on the indentation and by spatially orienting the adapter with the dispenser while the user is looking at the alignment indicator where the see-through channel will make it possible for the user to visually align the two elements 2, 3 of the alignment indicator, like the cross-hair pattern of a weapon, by spatially orienting the spray guide adapter 1 holding the bottom end 14 of the dispenser. When correctly aligned the user can activate the dispenser, like he/she normally uses the dispenser, and the spray guide adapter have thereby ensured that the correct application angle of the content of the dispenser inside the nostril of the user is used.

One example of a description of the operating steps with a spray guide adapter with possible adjustable target application angle:

Adjustment of the dispensing angle: before placing the nasal spray dispenser in the spray guide adapter 1, if necessary the user needs to adjust the dispensing angle by which the fluid of the nasal spray dispenser will be delivered. The angle is adjusted by changing the position and/or the line of sight of the alignment indicator in the nasal spray guide adapter 1. The angle is adjusted by rotating the rotation disk 12 containing at least 3 different angles.

Position of the nasal spray dispenser: The lower end 14 of a nasal spray dispenser is placed in the dispenser opening 4 of the spray guide adapter 1; the nasal spray dispenser is held by the protrusion 5 which is located on the bottom end of the dispenser opening 4.

Optimal angular position: by placing a thumb on the indentation 6 located in a bottom end 7 of the nasal spray guide adapter 1, the user holds the nasal spray guide adapter 1 containing the nasal spray dispenser 14, then the user inserts the elongated upper end of the nasal spray dispenser 15 inside one of the nostrils; while the elongated upper end of the nasal spray dispenser is in the nostril, the solid element 2 and the hollow element 3 of the alignment indicator are positioned to the left or right (depending on user preference) of the user's nose as visual feedback. The alignment indicator comprises a cross hair pattern with two elements, a solid element 2 and a hollow element 3; the solid element is a solid circle 2 located on the top surface 9 of the adapter 1; and the hollow element is a ring 3 located towards the bottom surface 7 of the adapter 1. Then the user proceeds to visually align the solid circle 2 with the hollow ring 3. After the cross hair alignment indicator is adjusted the user proceeds to activate the nasal spray dispenser to deliver to the nostril of the user the fluid in the nasal spray dispenser.

Assembly of the nasal spray guide adapter: if necessary, the spray guide adapter has to be assembled first by folding the adapter, as described above. The foldable adapter 10 can be provided as a plane element with folding lines 11 indicating where the folding needs to be performed.

FIGS. 9a-9f shows one embodiment of the presently disclosed spray guide adapter 91. All of FIGS. 9 a-9f show the same spray guide adapter 91, but from different view angles. A dispenser opening 94 for insertion of the dispenser is most easily seen in front perspective views in FIGS. 9a, 9c, 9e and 9f. An indentation 96 is provided in the bottom end of the adapter 91 to support a thumb of the user holding the adapter. The visual alignment indicator comprises an upper element 97 and a lower element 98, both extending radially from the dispenser opening 94. A visual element 92 is formed in upper element 97, the visual element 92 is an opening forming a channel through the upper element 97, i.e. a hollow visual element 92, which is best seen in FIGS. 9d, 9e and 9f. Two different visual elements 93, 93′ are formed on the lower element 98 as the letters “L” (reference numeral 93) and “R” (reference numeral 93′) indicating “Left” and “Right”, respectively. In FIG. 9e the adapter 91 is oriented such that visual element “R” in lower element 98 is visible through the hollow and transparent visual element 92 formed in the upper element 97. In FIG. 9f the adapter 91 is oriented such that visual element “L” in lower element 98 is visible through the hollow and transparent visual element 92 formed in the upper element 97. The point is that the combination of visual elements 92, 93, 93′ provides two different line of sights to the user during use of a nasal spray dispenser (not shown in FIGS. 9a-9f) located in the adapter 91: One line of sight for nasal spray distribution in the right nostril and another line of sight for distribution in the left nostril.

When the user have to apply spray in the right nostril, the user grabs the adapter 91 (with a dispenser in the opening 94), places his right thumb in the indentation 96 and the elongated upper end of the dispenser in the right nostril. The user then looks through the transparent visual element 92 and orients the adapter 91 with the dispenser until the user can see the letter “R” on the lower element 98. In that case the dispenser will be correctly aligned in the right nostril and user can activate the dispenser and the nasal spray will be efficiently and correctly distributed. When the user have to apply spray in the left nostril, the user can move the elongated upper end of the dispenser to the left nostril. The user then looks through the transparent visual element 92 and orients the adapter 91 with the dispenser until the user can see the letter “L” on the lower element 98. In that case the dispenser will be correctly aligned in the left nostril and user can activate the dispenser and the nasal spray will be efficiently and correctly distributed.

The embodiment illustrated in FIGS. 9a-9f provides for two different lines of sight for optimized nasal spray application in right and left nostril, respectively. However, the principle with the visual alignment indicator having radially extending elements for housing the various visual elements can also be used to define only a single line of sight, for example with a single visual element in the lower element 98, or more than two lines of sight, for example with more than two visual elements provided on the lower element 98. More than one line of sight can also be provided with more than one visual element in the upper element 97. And the embodiment in FIGS. 6a-6i provides an example with three different lines of sight provided by a rotational element 12.

As stated previously the present disclosure further relates to nasal spray dispenser comprising the presently disclosed alignment indicator as a fixed and/or integral part of the nasal spray dispenser. Features from the embodiment of the nasal spray adapter 91 shown in FIG. 9a-9f would be a good candidate for such a nasal spray dispenser. The upper element 97 and the lower element 98 extending radially from the dispenser opening 94 could be attached directly on a nasal spray dispenser (see example of a nasal spray dispenser in FIG. 7) such that the visual alignment indicator becomes an integral part of the dispenser without the need for an adapter. However, also the principles shown in FIGS. 1a-3d and 6a-7h, where the visual elements are part of a channel to function as a foresight, could be applied as fixed to and/or an integral part of a nasal spray dispenser.

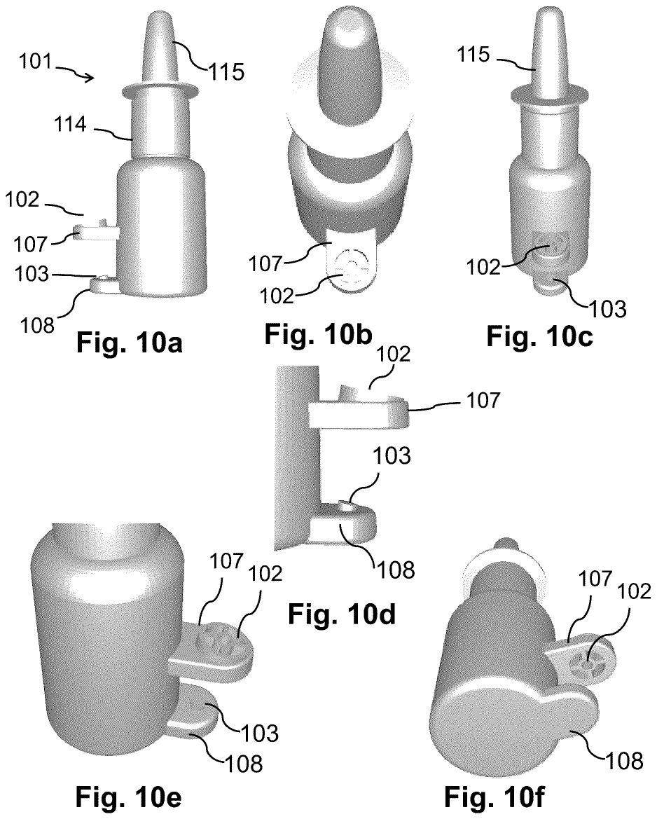

FIGS. 10a-10f show one embodiment of the presently disclosed nasal spray dispenser 101 with the presently disclosed alignment indicator as an integral part of the dispenser 101 having an elongated upper end 115. All of FIGS. 10a-10f show the same dispenser 101, but from different view angles.

The visual alignment indicator comprises an upper element 107 and a lower element 108, both extending radially from the dispenser 101. A visual element 102 is formed in upper element 107, the visual element 102 comprises an opening forming a channel through the upper element 107, i.e. a hollow visual element 102, which is best seen in FIG. 10f. The hollow visual element 102 comprises a cross-like structure with a small center opening such that the visual element 102 forms a front sight for a user of the dispenser 101. Another visual elements 103 is formed on the lower element 108. Visual element 103 is merely a small protrusion from the lower element 108, but it could also be a marking on lower element 108.

In FIG. 10b the dispenser 101 is oriented such that visual element 103 in lower element 108 in principle should be visible through the hollow and transparent visual element 102 formed in the upper element 107. In real life visual element 103 would be visible in FIG. 10b, but the actual representation in FIG. 10b makes it difficult to see the lower visual element 103 through the upper visual element 102. The lower visual element 103 provided in the lower element 108 can be provided with one more symbols, marking or colors such that it more clearly stands out from lower element 108. For example a small center dot in a contrasting colour to make it more visible to the user when aiming through the front sight 102.

FIG. 10d is a close-up sideview of the alignment indicator, where it can be seen that visual element 102 with the integrated front sight is slightly angled relative to the upper element 107. It can also be seen that upper element 107 extends further radially out from the dispenser than lower element 108, such that centers of visual elements 102, 103 are distanced differently from the dispenser (in the radial direction), with the lower visual element 103 being closer to the dispenser. The line of sight providing feedback to the user when the elongated upper end 115 is in a nostril of the user, is along the centers of visual elements 102, 103, and because these centers are aligned along another axis than the longitudinal axis of the dispenser, the alignment indicator in FIGS. 10a-10f forms an axis which is non-parallel with a longitudinal axis formed by the elongated upper end 115 of the dispenser 101, such that said longitudinal axis of the dispenser forms an alignment angle with a line of sight of the alignment indicator. This alignment angle defines the target application angle of the dispenser 101.

The embodiments and dispensing examples shown in the drawings are shown for right hand use. However, the same principle as herein described can equally well be applied to left hand use; in such case most of the features of the illustrated adapters could merely be mirrored to provide for left hand use.

1. An alignment indicator for a nasal spray dispenser, the nasal spray dispenser comprising a lower end and an elongated upper end for dispensing nasal spray fluid into a nostril of a user, the alignment indicator comprising at least two different visual elements configured for defining at least one line of sight for the user, thereby providing the user of the nasal spray dispenser with visual feedback on the orientation of the elongated upper end of the nasal spray dispenser in the nostril of the user, wherein a visual feedback is provided to the user when two of the visual elements are visually aligned to the user along one line of sight.

2. The alignment indicator according claim 1, wherein the alignment indicator comprises at least one channel for at least partly defining the line of sight.

3. The alignment indicator according to claim 2, wherein the diameter of the channel is less than 10 mm, preferably less than 8 mm, more preferably less than 6 mm, most preferably less than 4 mm, or even less than 3, or 2 or 1 mm.

4. The alignment indicator according to any of the preceding claims, configured to be a front sight for the user while orienting the upper end of the nasal spray dispenser in the nostril while holding the dispenser.

5. The adapter according to any of the preceding claims, wherein at least one of the visual elements are located towards an upper part of the alignment indicator and at least one other and different visual element is located towards a lower part of the alignment indicator.

6. The alignment indicator according to any of the preceding claims, wherein the visual alignment indicator extends longitudinally between at least two different visual elements.

7. The alignment indicator according to any of the preceding claims, wherein the alignment indicator comprises a cross-hair pattern for visual guidance of the user.

8. The alignment indicator according to any of the preceding claims, wherein the alignment indicator comprises a cross-hair pattern with a first end and a second end separated by a distance, the first end is located towards a top surface of the adapter; and the second end is located towards a bottom surface of the adapter.

9. The alignment indicator according to claim 8, wherein the cross-hair pattern comprises at least two visual element, such as at least two different visual elements, such as a solid element and a hollow element.

10. The alignment indicator according to any of preceding claims 8-9, wherein the cross-hair pattern comprises at least two visual element located at respective ends of the cross-hair pattern, such that a visual feedback is when the at least two elements are aligned to the user.

11. The alignment indicator according to any of preceding claims 8-10, wherein one visual element comprises a solid circle, and/or wherein another visual element comprises a ring, such as a circular ring.

12. The alignment indicator according to any of the preceding claims, wherein the visual elements comprise at least one solid element and at least one hollow element.

13. The alignment indicator according to any of the preceding claims, wherein the visual elements comprise at least two solid elements and at least one hollow element.

14. The alignment indicator according to claim 13, wherein the two solid elements are different, such as form different but recognizable symbols, such as different letters, like “R” and “L”, or different numbers.

15. The alignment indicator according to any of the preceding claims, wherein a visual feedback is provided when one of the visual elements, such as a solid visual element, is visible to the user through another of the visual elements, such as a hollow visual element.

16. The alignment indicator according to any of the preceding claims, wherein at least two of the different visual elements, such as a hollow element and a solid element, are separated along the line of sight and/or separated along a longitudinal axis defined by the nasal spray dispenser.

17. The alignment indicator according to 16, wherein the two different visual elements are separated by at least 10 mm, preferably by at least 20 mm, more preferably by at least 30, most preferably by at least 40 mm.

18. The alignment indicator according to any of the preceding claims, wherein the visual alignment indicator is configured for defining two different lines of sight for the user.

19. The alignment indicator according to claim 18, wherein the two different lines of sight are configured for orientation in the left nostril of the user and in the right nostril of the user, respectively.

20. The alignment indicator according to any of the preceding claims, wherein the visual alignment indicator is configured for visually indicating a first correct orientation in the left nostril of the user and a second correct orientation in the right nostril of the user.

21. The alignment indicator according to any of the preceding claims, wherein the visual elements comprise at least two solid elements

22. The alignment indicator according to any of the preceding claims, wherein at least two of the visual elements, such as two solid elements or two hollow elements, are separated in a plane defined by a bottom end of the dispenser.

23. The alignment indicator according to claim 22, wherein the visual elements are separated in the plane by at least 5 mm, preferably by at least 10 mm, more preferably by at least 15, most preferably by at least 20 mm.

24. The alignment indicator according to any of the preceding claims, wherein the visual alignment indicator comprises an upper element and a lower element, preferably extending radially from the dispenser, and wherein the visual elements are formed in said elements.

25. The alignment indicator according to any of the preceding claims, wherein the line of sight is adjustable.

26. The alignment indicator according to any of the preceding claims, wherein the alignment indicator comprises a rotatable element defining two or more different lines of sight, such that the line of sight is adjustable.

27. The alignment indicator according to any of the preceding claims, wherein the alignment indicator is a tactile alignment indicator configured for providing a tactile feedback to the user, such as sound, and/or vibration.

28. The alignment indicator according to any of the preceding claims, wherein the alignment indicator forms an axis which is non-parallel with a longitudinal axis formed by the elongated upper end of the dispenser, such that said longitudinal axis forms an alignment angle with a line of sight of the alignment indicator.

29. The alignment indicator according to claim 28, wherein the alignment angle is between 1 and 40 degrees, more preferably between 5 and 25 degrees, most preferably between 10 and 20 degrees, such as 15 degrees.

30. The alignment indicator according to any of the preceding claims, wherein the alignment indicator comprises a light source, for example located at a bottom of the channel.

31. The alignment indicator according to any of the preceding claims, wherein a distance between two of the visual elements along the line of sight is less than 40 mm, such a less than 30 or less than 20 mm.

32. The alignment indicator according to any of the preceding claims, wherein the delivery angle of the fluid of the nasal spray dispenser can be adjusted by modifying the position of the alignment indicator.

33. The alignment indicator according to any of the preceding claims, comprising at least one sensor configured for providing a tactile signal, such as a sound and/or vibration, and/or a visual signal, when the adapter is within a predefined spatial orientation.

34. A nasal spray dispenser comprising a lower end and an elongated upper end for dispensing nasal spray fluid into a nostril of a user, the nasal spray dispenser comprising an alignment indicator as defined in any one of the preceding claims.

35. The nasal spray dispenser according to claim 34, comprising an indentation for a finger, such as a thumb, of the user, for guiding the user when gripping the adapter, preferably the indentation is located in a bottom end of the dispenser.

36. The dispenser according to any of the preceding claims 34-35, wherein the fluid of the nasal spray dispenser is selected from the group of: Antihistamines, corticosteroids, saline and topical decongestants.

37. The dispenser according to any of the preceding claims 34-36, wherein the fluid of the nasal spray dispenser is a nasal vaccine.

38. The dispenser according to any of the preceding claims 34-37, wherein the alignment indicator is fixed to and/or is an integral part of the nasal spray dispenser.

39. A spray guide adapter for holding the nasal spray dispenser according to any of the preceding claims 34-37, the spray guide adapter, optionally comprising a housing, comprising a dispenser opening for inserting and holding the lower end of the nasal spray dispenser.

40. The adapter according to claim 39, wherein the alignment indicator is fixed to and/or is an integral part of the adapter.

41. The adapter according any one of the preceding claims 39-40, wherein the alignment indicator comprises at least one channel extending partly through the housing.

42. The adapter according any one of the preceding claims 39-41, wherein the alignment indicator comprises at least one channel extending fully through the housing, for example from the upper end to the lower end of the housing.

43. The adapter according to any of the preceding claims 39-42, wherein the diameter of the channel is less than 10 mm, preferably less than 8 mm, more preferably less than 6 mm, most preferably less than 4 mm, or even less than 3, or 2 or 1 mm.

44. The adapter according to any of the preceding claims 39-43, wherein the alignment indicator forms an axis which is non-parallel with an axial direction formed by the dispenser opening, such that when the nasal spray dispenser is held by the dispenser opening, a longitudinal axis formed by the elongated upper end of the nasal spray dispenser forms an alignment angle with a line of sight of the alignment indicator.

45. The adapter according to any of the preceding claims 39-44, wherein the dispenser opening is located in an upper end of the housing.

46. The adapter according to any of the preceding claims 39-45, wherein the housing comprises an indentation for a finger, such as a thumb, of the user, for guiding the user when gripping the adapter, preferably the indentation is located in a bottom end of the housing.

47. The adapter according to any of the preceding claims 39-46, wherein the dispenser opening comprises a protrusion for securing the lower end of the nasal spray dispenser.

48. The adapter according to claim 47, wherein the protrusion is located at an inner layer of the dispenser opening towards a center of the opening.

49. The adapter according to any of preceding claims 39-48, wherein the protrusion is located at the bottom end of the opening.

50. The adapter according to any of preceding claims 39-49, wherein a height of the housing is less than 40 mm, such a less than 30 or less than 20 mm, a width of the housing is less than 40, 30 or 20 mm, and/or a length of the housing is less than 40, 30 or 20 mm.

51. The adapter according to any of preceding claims 39-50, wherein the housing of the adapter is manufactured in polymer/plastic, metal, card board.

52. The adapter according to any of the preceding claims 39-51, wherein the housing is foldable and is provided as a substantially plane element with folding lines indicated.

53. The adapter according to claim 52, configured such that the housing is assembled by folding the plane element along the folding lines.

54. The adapter according to any of the preceding claims 52-53, wherein the housing is provided in card board.

55. The adapter according to any of preceding claims 39-54, wherein the adapter is incorporated as part of the packaging of the nasal spray dispenser.

56. A method of guided nasal spraying in a nostril of a user from a nasal spray dispenser comprising the steps of:
a. Placing the lower end of a nasal spray dispenser in the dispenser opening of the spray guide adapter according to any of the preceding claims 39-55 or
providing a nasal spray dispenser according to any one of claims 34-37 having a visual alignment indicator;
b. Grabbing by the user the adapter with the nasal spray dispenser attached or the nasal spray dispenser with the alignment indicator,
c. Inserting, by the user, the elongated upper end of the nasal spray dispenser inside the nostril of the user;
d. Angularly orientating by the user, while holding the nasal spray dispenser, the elongated upper end of the nasal spray dispenser inside the nostril of the user until the alignment indicator is aligned for the user by means of a visual and/or tactile feedback, and
e. Activating the nasal spray dispenser to deliver nasal spray to the nostril of the user.

57. The method according to claim 56, comprising, optionally before placing the nasal spray dispenser in the spray guided adapter, adjusting the dispensing angle of fluid of the nasal spray dispenser, by changing the position and/or the line of sight of the alignment indicator.

58. The method according to any of preceding claims 56-57, wherein the alignment indicator is aligned by the user aligning at least two elements of the alignment indicator, such as two elements of a cross-hair pattern.

59. The method according to any of preceding claims 56-58, wherein the spray guide adapter has to be assembled first by folding the foldable unit.
